(19) United States
(12) Patent Application Publication
(10) Pub. No.: US 2008/0097267 A1
(43) Pub. Date: Apr. 24, 2008

(54) Improved Orthotic Appliance for Carpal Tunnel Syndrome

(76) Inventor: George Roger Williams, Norman, OK (US)

Correspondence Address: DOERNER, SAUNDERS, DANIEL & ANDERSON, LLP, 201 Robert S. Kerr Avenue, Suite 700, OKLAHOMA CITY, OK, 73102-4203, US

(21) Appl. No.: 11/665,880

(22) PCT Filed: Oct. 20, 2005
(86) PCT No.: PCT/US05/38229

§ 371 Date: Apr. 20, 2007

Related U.S. Application Data
(60) Provisional application No. 60/605,674, filed on Oct. 20, 2004.

(51) Int. Cl. A61F 5/00

(52) U.S. Cl. 602/5; 602/21

(57) ABSTRACT

The present invention provides an orthotic appliance for the carpus of a human hand for the treatment of a carpal tunnel syndrome condition using co-dynamic, rather than traditional static or dynamic, techniques. The appliance may apply a dorsally-directed force to the region of the pisiform bone (23) in the neutral carpal position of a human hand upon co-contraction of the hand and up to 8 pounds dorsally-directed during arc of motion in carpal flexion, as the wrist and hand is encouraged to actively move in all planes of motion without restricting arc of motion or negatively affecting the normal activities of daily living. The appliance may comprise a biasing structure for applying the dorsally-directed force and a base structure for maintaining the biasing structure in its proper configuration during normal hand motion.

### CROSS REFERENCES TO RELATED APPLICATIONS

This application claims the benefit of U.S. Provisional Application No. 60/605,674, having a priority filing date of Oct. 20, 2004.

### TECHNICAL FIELD

The present invention relates generally to the field of orthotics and splints for the carpus of a human hand, and more particularly to an orthotic for the treatment of carpal tunnel syndrome using dynamic and co-dynamic, rather than static, techniques.

### BACKGROUND ART

Carpal Tunnel Syndrome (“CTS”) is a condition resulting from the compression of the median nerve that travels through an area in carpus of the hand between the carpal bones and a ligament known as the flexor retinaculum. This compression results in pain, numbness, and tingling in the hand and weakness in the grip. CTS often refers pain and paresthesia in the arm shoulders and neck.

There are many theories as to the causes of CTS. Some believe that it results from irritation of bursa, tendon sheaths, and nerve causing tunnel swelling from repetitive motion. Others attribute CTS to carpal fractures or arthritic joint changes. Still other schools of thought attribute CTS to systemic disease, mechanical stress, or traumatic dislocation. The compression theory that is widely accepted holds that irritated and inflamed tissue resulting from these events within the carpal tunnel compresses the median nerve within the confined space formed by the flexor retinaculum and the carpal bones. The inventor has investigated these causes and has formulated a Theory of Environmental Deformity (“TED”), which explains the causes and progression of carpal tunnel syndrome and suggests a method and apparatus for the resolution of these problems. U.S. Pat. No. 6,723,061, issued to the inventor and included herein by reference in its entirety, presents these causes, symptoms, and therapies under the TED.

Whereas conventional theories of CTS deal with the known movements of extension/flexion of the hand and wrist, radial/ulnar deviation, and supination/pronation, the approach incorporated in the TED addresses an additional observed movement called “glide”, which is superimposed over these known movements and has heretofore been unobserted. The TED thus addresses both the kinematics of the joint, or arthrokinematics, and the neuromuscular changes to explain how CTS arises and to suggest a treatment for the condition.

Note that during this discussion and throughout the remainder of the disclosure, the term “volar” shall be interpreted as “in the direction of the palm of the hand” and “dorsal” shall be interpreted as the opposite of “volar”, that is, in a direction away from the palm of the hand or directed outwardly from the back of the hand. Carpal extension/flexion occurs when the palm of the hand is moved upwardly and downwardly in a waving motion. This should be distinguished from digital extension/flexion, which occurs when the fingers of the hand are extended to their full range (“extension”) and when the hand and fingers are enclosed into a fist (“flexion”). Radial/ulnar deviation occurs when the palm is moved inwardly (radial deviation) and outwardly (ulnar deviation) about the end of the forearm without departing from the general plane of the radius or the ulna bones. Supination/pronation occurs when the hand and forearm is rotated between a palm up and a palm down attitude. Arthrokinematics is the science of the interplay between the dynamics and the soft tissue of a functioning joint. Unless specifically stated otherwise, all descriptions and observations shall be made from the standpoint of an individual's right hand and forearm for consistency and ease of description. The term “proximal” indicates a direction away from the elbow of the forearm, and the term “distal” indicates a direction towards or closest to the elbow. The discussion that follows applies equally well to either hand or forearm.

Referring to FIGS. 1 and 2, the normal human physiology exhibits a fixed cavity or area through which pass several tendons 44 and the median nerve 40. This cavity is designated as the carpal tunnel. It is defined anteriorly by the flexor retinaculum 46 and posteriorly by two sets of carpal bones. The proximal set of bones as viewed from a medial to lateral perspective is the pisiform 23, triquetrum 24, lunate 25, and scaphoid 26; this set of bones is designated as the proximal carpal row 36. The distal set of bones from a medial to lateral perspective is the hamate 31, capitate 32, trapezoidium 33, and trapezium 34; this set of bones is designated as the distal carpal row 35. The pisiform 23 is attached evenly by ligaments extending in nine directions, where these ligaments include the following: piso-hamate ligament; the piso-metacarpal ligament; the proximal band of the flexor retinaculum 46; the triangular fibrocartilage complex; the flexor carpi ulnaris; the anterior portion of the medial collateral ligament; the extensor retinaculum; the abductor digiti minimi; and the pisotriquetral cartilage.

The flexor retinaculum 46 attaches to the carpus on either side of its open ends and functions as the pulley of the carpal tunnel 42 for extrinsic hand muscles to communicate between the muscle origin about the elbow and the insertion point at the fingers, thumb, and wrist. The median nerve 40 lies between the flexor retinaculum 46 and the bundles of flexor tendons 44. Functionally, the flexor retinaculum 46 adds strength to the carpus and, through its pulley action, lends efficiency to the muscle tendon power of the hand.

In a normal wrist and forearm, the flexor muscle tendons of the volar forearm acting on the wrist, fingers, and thumb typically exert a collective pulley force four times that of the extensor muscle tendons in the dorsal forearm, which act to dorsally stabilize the same members of the wrist and hand in the course of coordinated activities. This interaction between the flexor muscles and the extensor muscles, termed “co-contraction”, holds the joint in a stable position, depicted in FIG. 3, during activity in cooperation with the carpal ligament integrity. CTS is not observed in such a stabilized joint. Co-contraction is maintained in hand and finger function and coordinated movement of the fingers and thumb until acted upon by resistance of the fingers in digital extension or flexion. The ratio of flexor to extensor forces in the forearm of a normal person is typically about 4:1 (“force couple”), and it remains relatively constant throughout life during ordinary work activities.

However, force couple changes can occur at some point in the life span of a person, for whatever reason, resulting in the condition illustrated in FIG. 4. If the intensity and duration of tasks requiring finger, thumb, and wrist function initiate hypertrophy of the flexors, then neuromuscular inhibition of the extensors may facilitate and sustain an inefficient ratio of agonist/antagonist function of the extrinsic muscular forces of the hand. This ratio difference causes and promotes a volar carpal translation (VCT) of the plane of the carpal-metacarpal complex with respect to the ulnar-radial plane; this movement is termed “volar glide”. Note that this translation is not a rotation about the axis of either carpal row or a rotation about the forearm carpal joint, but a shear movement in which the two planes are kept generally parallel but not coincident; in short, the two planes are not coplanar and separated by an increasing distance. This is shown in FIG. 4 by the shear displacement of centerline 50 of the carpal-metacarpal plane in a volar direction (indicated by arrows 60) from the centerline 55 of the ulnar-radial plane.

During exertion over time, hypertrophy of flexor muscle groups alters the biomechanics of the wrist and hand, so that co-contraction gradually increases volar carpal pulley forces, thus reducing the capacity and function of the extensor muscle tendon groups. Mechanical changes and neuromuscular subclinical pathology; referred to as proprioceptive dysfunction, is cited in literature as a contributing factor to joint destabilization. The result desired from any therapeutic intervention is to assist in normalizing the force couple and minimizing over-control, as in this case the extrinsic flexors of the hand. When the flexors become stronger and overly efficient, then the extrinsic extensors fail to function properly according to a well known neuromuscular process called “reciprocal inhibition”; this process is referred to as Sherington's Law in rehabilitation science. Over-control of the flexors, which occurs in contracting or co-contracting hand function, increases the force allocation over the flexor pulley (i.e. the flexor retinaculum 46), thus causing VCT in the direction indicated by arrows 60. Consequently, the long moment arm of the extensor carpi ulnaris, extensor radialis brevis, and the extensor carpi radialis longus, as well as the contribution of extensor communis, all eventually become inefficient in mechanical and neuromuscular physiology. This inefficiency, coupled with ligament length disparity, results in a lack of stabilization and involuntary VCT in the direction indicated by arrows 60. VCT becomes even more pronounced during digital extension, during which the luno-capitate joint is translated, i.e. the junction between the lunate and the capitate. The same holds true during digital flexion, during which the radial-lunate joint is translated, i.e. the junction between the lunate and the end of the radius of the forearm.

The effect of the volar translating flexor forces, acting upon the flexor retinaculum 46 as a pulley, attenuate the flexor retinaculum 46, and residual force distribution conveys forces anteriorlly and medially. This places traction forces to the ligament ends of the carpus. Each night, while the muscles are at rest, the volar intracarpal ligament segments restore their normal position grossly; however, some minute anteriomedial deformity remains, and slack of the flexor retinaculum 46 is concurrently taken up by contractile forces of this and volar intracarpal ligaments. Numerous cycles of force followed by rest develop and establish deformation that is manifested by narrowing the horseshoe ends of the carpal tunnel. The horseshoe ends are held in position by a clinically recognized, thickening flexor retinaculum 46 and other volar carpal ligaments, resulting in a transverse deformity. Simultaneously, the flexor retinaculum 46 acting as a pulley is subjected to the load produced by the finger and thumb function through digital extension/flexion, so that VCT increases along with the VCTF that accompanies VCT. Thus, laxity of the dorsal carpal ligaments originating from the distal radial ulna increases. The volar carpal ligaments (including the flexor retinaculum 46) collectively become stressed intermittently and thus contract (shorten), which encourages the anteriomedial collapse (diminished carpal volume) of the intercarpal and intracarpal spaces simultaneously with longitudinal deformity, with continued VCT promoting an obtuse canal or “Guillotine” effect of the median nerve at the wrist.

The long moment arm of the carpal muscle tendon units are only capable of stabilization of the carpus when the muscle tone is within normal limits, i.e. flexor to extensor force ratio of approximately 4:1. These forces acting on the carpus in flexion are convergent toward the muscle origin and are regulated by interplay of antagonists, pulleys, and joint alignment. A variation of one or more serves to simplify convergence towards a direct line to this point of origin and shorten the distance therebetween. This resulting force decreases the biomechanical advantage, manifested by a volar shift of the axis of the proximal carpal row 36 and distal carpal row 35 in a shear, or gliding, movement. The volume of the carpal tunnel 42 is further reduced thereby, and any other abnormal predisposition will hasten onset of the condition. Thus, the resistance that the flexor retinaculum 46 and related volar ligaments encounter when returning the carpus to a neutral position, i.e. dorsal glide, is indicative of the severity of CTS or the propensity of the subject to incur the condition in an otherwise normal wrist.

In order to restore normal carpus and hand function in CTS patients, carpal stabilization must be achieved. Carpal stabilization is believed to depend largely upon neuromuscular and proprioceptive control, a concept that is absent from conservative methods of managing CTS. Carpal stabilization consists of restoring the normal force couple of the flexors and extensors in the forearm.

Static splinting of the carpus and hand has been employed in the prior art to relieve CTS symptoms, but such relief is only temporary. Although static splinting positions the carpus so that the flexors and extensors may properly apply their forces to the carpal area, the fundamental imbalance of forces remains; muscles atrophy, range of motion is lost, and no lasting clinical benefit has been documented when static splints are removed. Flexor over-control simply reestablishes the original series of events leading up to compression neuropathy of the carpus. Static splinting thus does not permanently restore proprioceptive control and normal arthrokinematics, and it can only provide temporary relief of symptoms. Furthermore, static splinting does not address the glide movement found during digital flexion/extension. The static splinting thus impairs proprioceptive control and results in further dysfunctional arthrokinematic conditions maintained by abnormal force couple.

One such static splinting approach is suggested by U.S. Pat. No. 5,868,692, issued to Michniewicz, which discloses a static wire conforming to the surface of the hand, carpus, and forearm that restricts a user's pronation and supination to 10°, thus preventing extreme torsion that is believed to aggravate those patients with prior arm and wrist injuries. Michniewicz does offer a more comfortable and less confining device than some of the other static splints, but does not address the gliding movement of the carpus with respect to the forearm.

Dynamic splinting that allows exercise of the flexors and extensors of the forearm has been found to be more effective than static splinting. However, dynamic splinting found in the prior art, as such, also does not address the glide movement of the hand during digital extension/flexion. Such prior art is only concerned with extension/flexion and/or radial/ulnar deviation by wrapping to compress it, forcing movement, or preventing movement of the carpus and hand in its available arc of motion.

Several devices are illustrative of this dynamic splinting approach. U.S. Pat. No. 5,653,680, issued to Cruz, discloses a device that dynamically controls flexion and extension of the wrist, and ulnar and radial deviations with adjustable damping springs, which appear to effectively limit active range of motion. The device applies rotational force to the wrist joint while pressuring to the second and third metacarpal bones, the pressure promoting a volar or dorsal transrelocation of the distal carpal row. By concentrating on the distal carpal row, Cruz places importance on independently pressuring a region removed from the carpal complex. Cruz further concentrates on the damping aspect of the invention, which is primarily directed to protect the joint against injuries due to shock than to prevent or correct a CTS condition. However, Cruz does not address volar glide during digital extension/flexion.

U.S. Pat. No. 5,413,553, issued to Downes, describes another device called a Carpal Tunnel Mitt that concentrates a mechanical opposition upon the 1st to 5th metacarpal-phalangeal region. The Carpal Tunnel Mitt is structured to deepen the carpal tunnel for decompression purposes and is distal to the actual flexion-extension mechanics occurring at the radio-carpal and mid-carpal region. Again, Downes does not address volar glide during digital extension/flexion.

U.S. Pat. No. 6,238,358, issued to Philot et al., discloses a reconfigurable multi-purpose orthopedic fixation device for use alternatively as an external orthopedic fixation device for providing support and/or traction for a sprained, fractured or broken limb of a person. The shape of the orthotic may be changed to accommodate each individual's ergonomic and anatomic aspect. This change of shape is therefore a static operation made by the therapist and not a dynamic operation that is volitional by the patient. It is described as an orthopedic fixation device providing fixation and support that is not dynamic, but only provides fixed support while allowing certain movements.

In addressing carpal stabilization, the TED identifies a bone of major importance in the proximal carpal row 36, i.e. the pisiform 23. As previously discussed, the pisiform 23 functions as the attachment point for support ligaments in nine directions. As flexor retinaculum 46 and volar intra-carpal ligaments undergo dysfunctional changes associated with CTS, the VCT increases, leaving the pisiform 23 susceptible to deformation by altered particular attachments thereto. In cases where CTS is severe, the pisiform 23 often succumbs to osteoarthritis and becomes immobilized. When the pisiform 23 is immobilized, the piso-triquetral joint 48 (the joint between the pisiform 23 and the triquetrum 24 in the proximal carpal row 36) is unable to produce proximal excursion during co-contraction and distal excursion during end range composite flexion.

The TED identifies at least three different types of dysfunction due to VCT affecting the moment arm of the pisiform 23. These types are based on the classification of the nine attachments to the pisiform 23 as being either distal pisiform attachments or proximal pisiform attachments. They are documented in the abstract paper presentation to the American Society for Peripheral Nerve, entitled “Pisiform Arthrokinematics and Carpal Tunnel Syndrome,” by G. R. Williams, p. 645 Vol. 18, No. 7, October 2002, Journal of Reconstructive Microsurgery, which is included herein by reference in its entirety. In summary, Type I pisiform behavior is caused by excessive grasping of the hand to produce contraction of the distal pisiform attachments. Type II pisiform behavior is caused by excessive co-contracting in digital extension of the hand to produce contraction (or shortening) of the proximal pisiform attachments. Type III pisiform behavior is caused by a combination of excessive contracting and co-contracting activities to produce multi-planar immobilization of the pisiform. A lack of pisiform mobility due to Type I or Type II displacement or Type III immobilization is intrinsically linked to intracarpal pulley forces translating the wrist (VCT) in excessive volar glide.

The TED focuses generally upon the causative effect of volar glide and the resultant specific to the limited excursion of the piso-triquetral joint 48 as a major arthrokinematic indicator of CTS wrists. The TED thus proposes a treatment to dynamically encourage realignment of the carpus, mobilization of the pisiform, and mobilization and stabilization of the forearm carpal joint. To do this, the TED identifies a plane of motion critical to treating CTS called dorsal glide. Dorsal glide, the opposite of volar glide, is the dorsal movement of the plane of the carpal metacarpal complex in a shear movement keeping it parallel with the plane formed by the forearm, radius, and ulna; such movement occurs at the proximal and distal carpal row 36. According to the TED, a continuous encouragement of dorsal glide will reestablish normal carpal height while maintaining and promoting normal range of movement in all other planes of motion involved in the standard activities of daily living. The preferred location to apply force encouraging dorsal glide is generally in the region of the pisiform 23 leveraging the proximal and distal carpal rows 35, 36. Through an interactive, dynamic, resistance-oriented application of dorsal glide force at the pisiform 23 and pisiform region, the TED addresses both movement (kinematics) and control (neuromuscular) aspects treating CTS. Previous solutions have concentrated upon the flexion/extension, ulnar/radial deviation, and supination/pronation ranges of motion (i.e. rotational) but have neglected the valuable contribution of dorsal and volar glide (i.e. translational) involved in and critical to the normal arthrokinematics of carpal and hand function.

CTS and its related disorders are responsible for very high corporate overhead in terms of lost productivity, worker's compensation, and related medical costs from having to subsequently treat the condition that often becomes chronic due to the traditional paradigm of CTS diagnosis and treatment.

As can be seen, there is a need for an orthotic appliance that more precisely addresses the root causes and symptoms of CTS, according to the TED. There is a further need for the orthotic appliance to be inexpensive and easy to fit and use by a layperson. The appliance should be co-dynamic so that it works cooperatively with the hand and carpus to achieve the therapeutic result of correcting a CTS condition. It should allow the hand and wrist to move functionally in all planes of motion so as not to interfere with the normal activities of daily living and to address the condition in reverse by the individual choice of hand behaviors, thereby correcting the underlying causes. This specific and individual hand use under codynamic interference has been documented in preliminary research to increase carpal tunnel dimensions, thus reducing intracarpal pressure.

### SUMMARY OF THE INVENTION

The present invention achieves its intended purposes, objects, and advantages through a new, useful, and unobvious combination of component elements, with the use of a minimum number of functioning parts, at a reasonable cost to manufacture, and by employing only readily available materials. In these respects, the present version of the invention substantially departs from the conventional concepts and designs of the prior art, and in so doing, provides an apparatus that substantially fulfills this need. Additionally, the prior patents and commercial techniques do not suggest the present inventive combination of component elements arranged and configured as disclosed herein.

In one aspect of the invention, an orthotic appliance is provided for the treatment of a carpal tunnel syndrome condition in a carpus and a hand of a person, the carpus and hand exhibiting volar glide during digital extension of fingers of the hand. The appliance may comprise a biasing component having a first end and a second end, the second end adapted for positioning on the volar surface of the hand in the region of the pisiform bone, the biasing component positioned so that the second end applies a dorsally-directed force leveraged against the first end, wherein the dorsally-directed force opposes the volar carpal translation force manifested by the carpal tunnel syndrome condition during digital extension/flexion and disposed to urge the distal carpal row to move in a dorsal direction; and a base component supporting the biasing component.

In another aspect of the invention, a method of relieving carpal tunnel syndrome condition exhibited by a person having a hand, a carpus, and a forearm is provided, the carpal tunnel syndrome condition having produced a volar translation of the carpal-metacarpal complex in excess of the volar translation inherent in a normally-functioning person. The method may comprise the steps of providing a biasing component adapted to apply a dorsally-directed force to the general region of the pisiform bone of the carpus; arranging the biasing component so that the dorsally-directed force is applied in opposition to a volar carpal translation force that occurs during voluntary digital extension/flexion of the fingers of the hand; and maintaining the biasing component along the surface of the hand, carpus, and forearm during normal activities of daily living.

These and other features, aspects and advantages of the present invention will become better understood with reference to the following drawings, description, and claims. For a better understanding of the invention, its operating advantages and the specific aspects of its uses, reference should be made to the accompanying drawings and descriptive matter in which there are illustrated preferred embodiments of the invention. The foregoing has outlined some of the more pertinent aspects of the invention. These aspects should be construed to be merely illustrative of some of the more prominent features and applications of the present invention. Many other beneficial results can be attained by applying the disclosed invention in a different manner or by modifying the invention within the scope of the disclosure. Accordingly, a better understanding of the invention will be promoted, and the detailed description of the preferred embodiments in addition to the scope of the invention will be illustrated by the accompanying drawings.

### BRIEF DESCRIPTION OF THE DRAWINGS

FIG. 1 shows a palmer view of a human hand with the surface removed to illustrate the relationship of the bones of the forearm, carpus, and hand;

FIG. 2 shows a cross sectional view of the carpal region of a right human hand and illustrates the alignment of the proximal carpal row with relationship to the flexor retinaculum;

FIG. 3 shows an ulnar view of a unsymptomatic hand and carpus having a normal arrangement;

FIG. 4 shows an ulnar view of a hand and carpus of a patient with symptoms of CTS, the proximal carpal row being translated in a volar direction;

FIG. 5 shows a dorsal view of a hand, carpus, and forearm of a person with an embodiment of the invention installed thereon; and

FIG. 6 shows a volar, perspective view of a hand, carpus, and forearm of a person with an embodiment of the invention installed thereon.

### BEST MODES OF CARRYING OUT THE INVENTION

The following detailed description shows the best currently contemplated modes of carrying out the invention. The description is not to be taken in a limiting sense, but is made for the purpose of illustrating the general principles of the invention and the best mode for practicing the invention, since the scope of the invention is best defined by the appended claims.

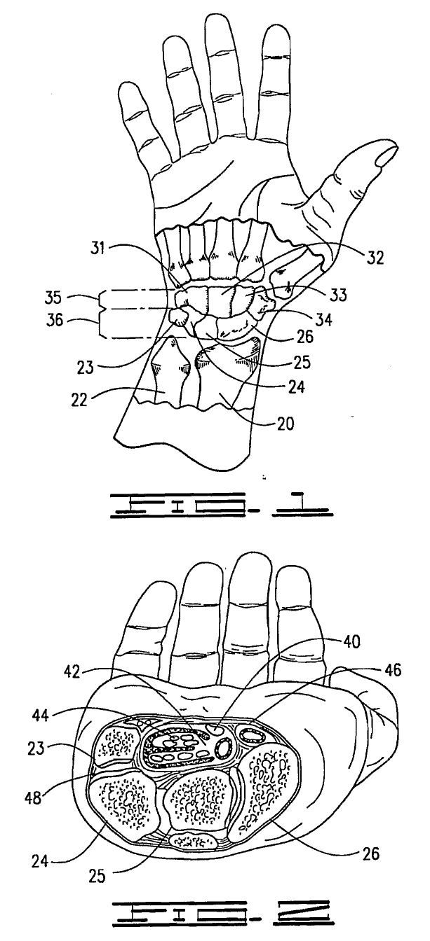

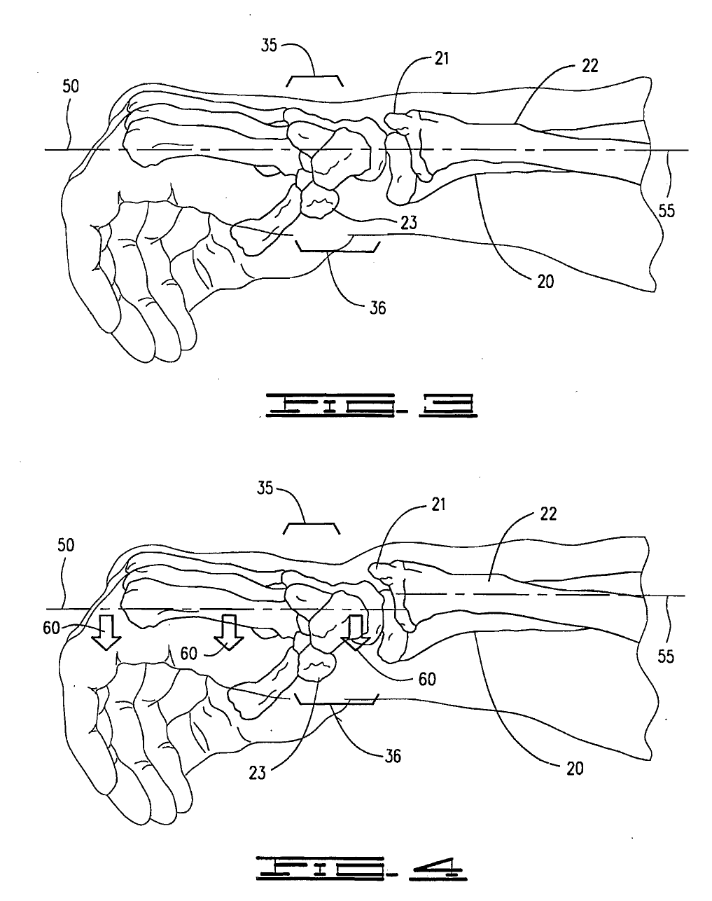

The invention comprises a dynamic orthotic device and method for the correction of CTS, wherein the device opposes volar glide during digital extension/flexion without inhibiting movement the hand, carpus, and forearm during normal activities of daily living. The person may perform extension/flexion of the carpus and hand, supination/pronation of the forearm, and ulnar/radial deviation of the hand without restriction. The prior art focuses on providing resistance to flexion/extension of the carpus, a rotational movement about the carpus, but it fails to address VCT during digital flexion/extension. When a person with CTS either extends the fingers (“digital extension”) or makes a fist (“digital flexion”), volar glide occurs without obvious flexion or extension of the carpus. The dynamic orthotic device directs resistance to volar glide, unlike the dynamic splints in the prior art. Furthermore, the dynamic orthotic device interactively provides a dynamic force directly to the pisiform region of the carpus in order to oppose VCTF during digital extension/flexion and to thus encourage dorsal glide in a human hand. The interactive, dynamic orthotic appliance provided by the invention provides dynamic resistance against volar glide (excessive in CTS patients) of the proximal and distal carpal row 36 (FIGS. 1 and 4) and thus promotes dorsal glide throughout the normal range of motion of a human hand during normal activities of daily living. The orthotic appliance may utilize a biasing component to communicate a dorsally-directed force against a point of leverage to dorsally translate the carpal-metacarpal complex in a shear movement. A preferred point of leverage identified by the TED is in the region of the pisiform 23, i.e. the general area of the hand that is either directly on the pisiform 23 or somewhat distal to the pisiform along the ulnar side of the palm. The goal is to minimize or eliminate any interference with normal hand and wrist motion while applying an as-needed, self-initiated counterforce opposing VCT to the pisiform region of the hand in order to achieve a dorsal realignment of the carpus with respect to the forearm, i.e. to promote dorsal glide, and to maintain that alignment by eccentric input of the weaker extensor muscle tendon groups and, in so doing, avoid external pressure over the carpal canal. Dorsally-directed force of as little as 1½ lbs., determined by applied hand activity (i.e. digital extension/flexion), may allow unrestricted, multiplanar use of the hand, yet eliminates symptoms typically within 24 hours while using the hand and arm in unfettered activities of daily living. Since during digital extension the luno-capitate joint undergoes translation, then the dorsally-directed force is applied to the distal carpal row for optimal results; since during digital flexion the radial-lunate joint undergoes translation, then the dorsally-directed force is applied to the proximal carpal row for optimal results.

The invention provides a unique biasing component for applying the dorsally-directed force to the pisiform region, where the biasing component may be a resilient wire formed as an elastic spring. Unlike the inventor's own prior art in which both portions, referenced as “ends” in the prior art, of the biasing component are stabilized generally along the dorsal surface of the forearm, the dynamic orthotic device of the present invention may employ a resilient wire having only one portion stabilized along the dorsal forearm. The other portion may be wrapped around and universalized to the ulnar side of the hand in such a way as to avoid interference with normal activities of daily living and made to terminate in the general pisiform region. Such a configuration may allow for expansion to permit a one-size-fits-all, self-conforming component. This resilient wire may be held in conformance with the contour of the hand by a base component that is less bulky than a base component of the prior art, the present base component serving merely as a covering for the biasing component and an attachment platform for straps holding it in conforming relationship with the person's hand, carpus, and forearm. The elastic spring and base component may be constructed of materials that may be easily washed for extended use.

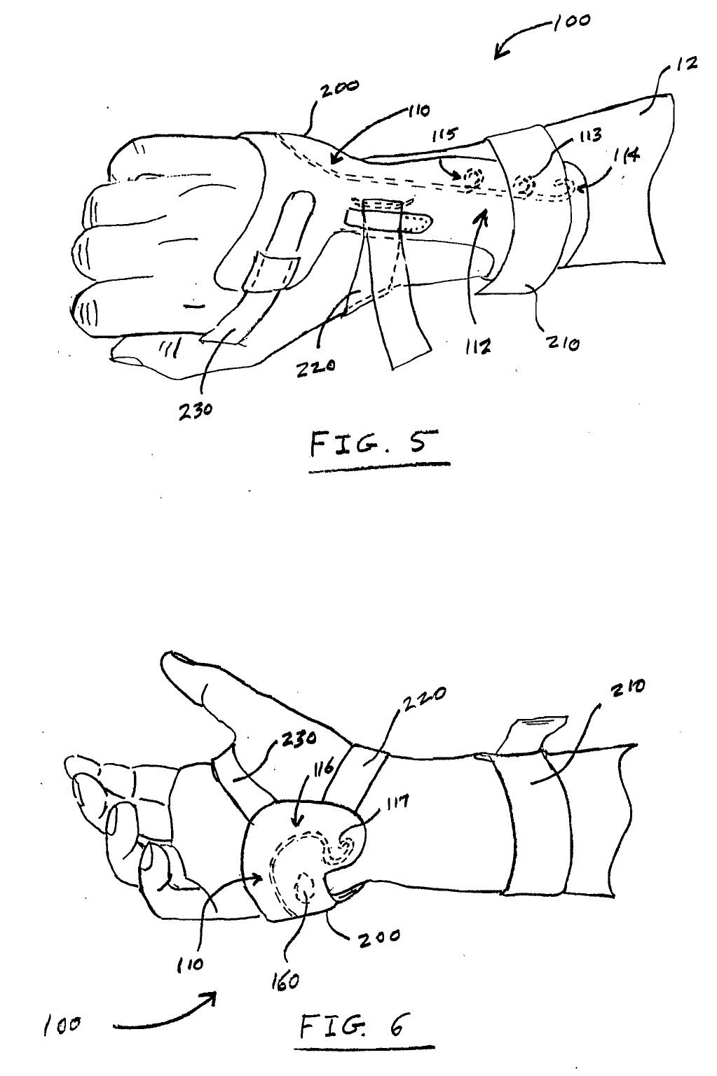

Referring now to FIGS. 5 and 6, an embodiment of the dynamic orthotic appliance 100 is shown as configured on the hand, carpus, and forearm of a person having CTS. The appliance may comprise a biasing component 110 made of a resilient material and formed to provide a relatively straight first portion 112 that may be generally aligned along the dorsum 120 of the forearm and a curved second portion 113 that may be generally aligned about the pisiform region 160 of the carpus where the pisiform bone 23 is located. The first portion 112 may terminate at a first end 122, and the second portion 113 may terminate at a second end 132.

The biasing component 110 may be held by a base component 200 in a conforming relationship with the dorsum, ulnar side of the hand, and the pisiform region 160 of the hand. The base component 200 may be removably attached to the forearm by a forearm strap 210 with one end fixedly attached to a proximal portion of the base component 200. It may also be removably attached to the carpus by a proximal thenar strap 220 and to the hand by a distal thenar strap 230, both straps with one end fixedly attached to a distal portion of the base component 200. The base component 200 may have sufficient flexibility to allow the carpus to extend and flex through its normal range of motion. When the carpus is voluntarily extended (carpal extension) by the person wearing the device, the appliance may be configured to allow the first end 113 to move proximally to a position indicated by numeral 114; when the carpus is voluntarily flexed (carpal flexion), the appliance may be configured to allow the first end 113 to move distally to a position indicated by numeral 115. During such proximal and distal movement of the first end 113, the first portion 112 may be kept in conforming relationship with the surface of the dorsum 120 by the base component 200 without inhibiting such proximal/distal movement. The base component 200 may maintain the second portion 116 of the biasing component 110 in conforming relationship with the pisiform region 160 of the hand; this may be accomplished by coordinated adjustment of the proximal and distal thenar straps 220, 230 according to the size and shape of the hand. The first portion 112 thus serves as a stable fulcrum against which the second portion 116 is leveraged to provide a dorsally-directed force acting against VCTF, while the first end 113 is allowed to move distally and proximally to accommodate extension and flexion of the carpus.

The biasing component 110 may be fabricated out of any resilient material that is conformable to the contour of a human hand, carpus, and forearm but will hold its shape. According to the embodiment shown, a resilient wire may be used, but other materials may also be used, such as a metallic strip, graphite fibers, and the like. It has been found that a resilient wire may be easily cut and fabricated for use as a biasing component without elaborate tools or machinery, and it has the virtue of being simple. Combinations of materials, such as a metal band fabricated as the second portion, coupled with a resilient wire fabricated as the first portion, may also be used. However, other resilient materials may be used without departing from the scope of the invention.

The first end 113 of the biasing component 110 may be treated in such a way so as not to catch or bind during proximal/distal movement although a fixed first 113 would allow intended operation. In the embodiment shown, the first end 113 has a curved aspect in the case of a resilient wire, but other methods may be used without departing from the scope of the invention. For example, the first end 113 may be treated with a smooth epoxy button that slides easily along the material of the base component, or a tube (not shown) may be provided through which the first end travels without making contact with the material of the base component. Other methods of preventing binding and snagging may be used without departing from the scope of the invention.

As can be seen, only the first portion 113, and not both portions, of the biasing component 110 may be aligned and attached to the forearm, whereas the prior art disclosed both portions being aligned and attached to the forearm. The singly-leveraged attachment of only one portion to the forearm may allow the biasing component 110 to provide a counterforce of as little as 6% of the highest values of VCTF observed in a person having CTS, as opposed to an 18% counterforce that has been previously assumed to be necessary to effect CTS correction. The presence of a singly-leveraged portion may also allow greater freedom of movement than a doubly-leveraged biasing component. It has been found through experimentation that as much as 18% of the VCTF is appropriate for so-called traumatic carpal tunnel syndrome (i.e. falling on the hand, crush injuries, capsule tears, etc.). Environmental carpal tunnel syndrome without trauma only requires 6% of VCTF to manage and stabilize the lunate and capitate in a majority of the cases. This requires less of a leveraging action by the biasing component 110 than heretofore, and the single lever arm of the present dynamic orthotic appliance, i.e. the first portion 113, is adequate for these reduced force requirements, as opposed to the dual lever arm arrangement of the prior art. Any dorsally-directed counterforce of between about 6% and about 18% of the VCTF exhibited by a person having CTS may be considered to be within the scope of the invention, with counterforce values towards the lower end of this range being more preferable, and a value of about 6% being most preferable.

The spring on the dorsum of the forearm, rests in approximately the same position. The spring, however, is far more efficient in that, before it makes its turn around the ulnar aspect of the palm, it attaches to elastic straps of precise efficiency from the opposing side coming from the volar aspect of the hand that adjusts it precisely to the surface of the person's hand and carpus and thus bring the three point stability from spring action into proper function, and the distal thenar strap is adjustable so that we can bring the spring in further stabilization to the palm from the volar aspect and attached to the dorsum of the orthosis by standard hook-and-loop means. The dynamic orthotic appliance having the means of expansion when the hand performs its activities in allowing for muscle contraction and differences in relative position of the hand throughout its arc of motion with that of the wrist, is truly co-dynamic in that it does not interfere with normal planar function of the wrist and hand.

The base component 110 may be constructed of any flexible material found in the art. Typically, the base component 110 may be constructed of two pieces of a flexible cloth material that are stitched around the biasing component 110 held therebetweeen. Additional stitching (not shown) may be provided to hold the biasing component in a fixed position between the two pieces and additionally to guide the first portion during carpal extension/flexion.

Other materials, such as a flexible plastic shell, hard rubber, or segmented metal sleeves, for example, may be used to fabricate the base component 110 without departing from the scope of the invention, but it has been found that a cloth material, such as rubber, Spandex™, Lycra™, neoprene, other elastometric materials, and the like, have advantages of simple fabrication, flexibility, and ability to be easily washed without damage.

The adjustable forearm strap 210, proximal thenar strap 220, and distal thenar strap 230 may similarly be constructed of any material typically found in the art, such as elastic strips, rubber, leather, synthetic materials, etc. It has been found that an elastic strip having a standard hook-and-eye fastener on the end portion of the strap provides easy installation and adjustment of the appliance. A typical hook-and-eye fastener may be Velcro™. When a hook-and-eye arrangement is used, the surface of the base component 110 may be covered with the eye portion of the hook-and-eye device, and the hook portion may be sewn to the strap. Other types of fasteners may be used to secure the straps without departing from the scope of the invention, such as snaps, buckles, adhesive tape, etc.

In operation, the dynamic orthotic appliance 100 may be fitted to a person's forearm, carpus, and hand, so that the first portion 112 of the biasing component 110 is aligned along the dorsum 120 of the forearm and the second portion 116 of the biasing component 110 is aligned over the pisiform region 160 of the carpus. When the first and second portions 112, 116 are thus aligned, the forearm strap 210 may be tightened to hold the base component 110 in a fixed position along the forearm, and the proximal thenar strap 220 and distal thenar strap 230 may be adjusted so that the base component 110 holds the second portion 116 of the biasing component 110 in a relatively fixed position over the pisiform region 160. Once attached to the forearm, carpus, and hand in this manner, the person may move the hand and carpus through any of the standard movements thereof, i.e. carpal extension/flexion, ulnar/radial deviation, and supination/pronation. When the person extends or bends the fingers (digital extension/flexion), the second portion of the biasing component is positioned to resist volar glide of the carpus, thereby improving the alignment by minimizing the effect of excessive VCTF and thus reestablishing the force couple between the extensors and flexors of the forearm.

As has been demonstrated the present invention provides an advantageous appliance and technique for prevention and correction of carpal tunnel syndrome within a human carpal joint. While the preferred embodiments of the present invention have been described, additional variations and modifications in those embodiments may occur to those skilled in the art once they learn of the basic inventive concepts. Therefore, it is intended that the appended claims shall be construed to include both the preferred embodiment and all such variations and modifications as fall within the spirit and scope of the invention.

1. An orthotic appliance for the treatment of a carpal tunnel syndrome condition in a carpus and a hand of a person, the carpus and hand exhibiting volar glide during digital extension of fingers of the hand, the appliance comprising
a biasing component (110) having a first portion (112) and a second portion (116), the second portion (116) adapted for positioning on the volar surface of the hand in the region of the pisiform bone (23), the biasing component (110) positioned so that the second portion (116) applies a dorsally-directed force leveraged against the first portion (112), wherein the dorsally-directed force opposes the volar carpal translation force during digital extension/flexion; and
a base component (200) supporting the biasing component (110).

2. The appliance described in claim 1, wherein the base component (200) is adapted to maintain the biasing component (110) in conforming relationship with a surface of the hand and the forearm throughout all activities of daily living.

3. The appliance described in claim 1, wherein the first portion (112) is adapted for positioning along the dorsal surface of the forearm supporting the hand, the first portion (112) moving distally and proximally along the forearm during extension and flexion of the hand.

4. The appliance described in claim 1, wherein the second portion (116) is adapted for positioning on the volar surface of the hand in the region (160) of the pisiform bone (23).

5. The appliance described in claim 1, wherein the base component (200) is constructed of an elastometric material.

6. The appliance described in claim 5, wherein the elastometric material is chosen from the group consisting of rubber, Lycra™, Spandex™, and Neoprene™.

7. The appliance described in claim 1, wherein the base component (200) is a flexible shell held in place by at least one strap (210, 220, 230), wherein the strap (210, 220, 230) is adapted to position and maintain alignment of the biasing component (110) along the surface of the forearm, hand, and carpus during extension/flexion of the hand, supination/pronation of the forearm, and ulnar/radial deviation of the hand.

8. The appliance in claim 1, wherein the biasing component (110) comprises a resilient material.

9. The appliance in claim 8, wherein the resilient material is a resilient wire.

10. The appliance in claim 8, wherein the resilient material is a metal strip.

11. The appliance in claim 8, wherein the resilient material is a strip comprised of a resilient fabric.

12. The appliance in claim 1, wherein the magnitude of the dorsally-directed force is about 6% of the magnitude of the volar carpal translation force during digital extension of the fingers and thumb when the carpus is in a neutral position.

13. The appliance in claim 1, wherein the magnitude of the dorsally-directed force is within a range of about 1½ lbs. to about 4 lbs.

14. An orthotic appliance for the treatment of a carpal tunnel syndrome condition in a carpus and a hand of a person, the carpus and hand exhibiting volar glide during digital extension of fingers of the hand, the appliance comprising
a resilient wire with a first portion (112) adapted for positioning along the dorsal surface of the forearm supporting the hand, the first portion (112) moving distally and proximally along the forearm during extension and flexion of the hand, the biasing component having a second portion (116) adapted for positioning on the volar surface of the hand in the region (160) of the pisiform bone (23), the resilient wire positioned so that the second portion (116) applies a dorsally-directed force leveraged against the first portion (112), wherein the dorsally-directed force opposes the volar carpal translation force during digital extension/flexion; and
a base component (200) maintaining the resilient wire in conforming relationship with a surface of the hand and the forearm throughout all activities of daily living.

15. The appliance in claim 14, wherein the base component (200) has a distal end and a proximal end.

16. The appliance in claim 15, wherein the proximal end is removably attached to the forearm by a forearm strap (210).

17. The appliance in claim 15, wherein the distal end is removably attached to the hand by a proximal thenar strap (220) and a distal thenar strap (230).

18. The appliance in claim 15, wherein the dorsally-directed force is directed against the distal carpal row (35) during digital extension.

19. The appliance in claim 15, wherein the dorsally-directed force is directed against the proximal carpal row (36) during digital flexion.

20. A method of relieving carpal tunnel syndrome condition exhibited by a person with a hand, a carpus, and a forearm, the carpal tunnel syndrome condition having produced a volar translation of the carpal-metacarpal joint of the carpus in excess of the volar translation inherent in a normally-functioning person, the method comprising the steps of
providing a biasing component (110) adapted to apply a dorsally-directed force to the general region (160) of the pisiform bone (23) of the carpus;
arranging the biasing component (110) so that the dorsally-directed force is applied in opposition to a volar carpal translation force that occurs during digital extension/flexion of the fingers of the hand; and
maintaining the biasing component (110) along the surface of the hand, carpus, and forearm during normal activities of daily living.

21. The method described in claim 20, wherein the dorsally-directed force is between about 1½ lbs and about 4 lbs.

22. The method described in claim 20, wherein the dorsally-directed force is in a range of about 6% of the volar carpal translation force to about 18% of the volar carpal translation force.

23. The method described in claim 20, wherein the dorsally-directed force is provided by a resilient wire a resilient wire with a first portion (112) adapted for positioning along the dorsum of the forearm supporting the hand, a first end (113) of the first portion (112) moving distally and proximally along the forearm during extension and flexion of the hand, the biasing component having a second portion (116) adapted for positioning on the volar surface of the hand in the region (160) of the pisiform bone (23), the biasing component positioned so that the second portion (116) applies a dorsally-directed force leveraged against the first portion (112), wherein the dorsally-directed force opposes the volar carpal translation force during digital extension/flexion, and wherein the dorsally-directed force is disposed to urge the distal and proximal carpal rows to move in a dorsal direction.
